(19) United States
(12) Patent Application Publication
(10) Pub. No.: US 2021/0269850 A1
(43) Pub. Date: Sep. 2, 2021

(54) DRY COMPOSITIONS FOR USE IN NUCLEIC ACID AMPLIFICATION AND METHODS

(71) Applicant: BIOFORTUNA LIMITED, Bromborough, Wirral (GB)

(72) Inventors: Kevin Lee, Bebington (GB); Michael Bunce, Caerwys (GB)

(21) Appl. No.: 16/756,198

(22) PCT Filed: Oct. 16, 2018
(86) PCT No.: PCT/GB2018/052974

§ 371 Date: Apr. 15, 2020

(30) Foreign Application Priority Data
Oct. 16, 2017 (GB) 1716963.2

(51) Int. Cl. C12Q 1/6806; C12Q 1/6804

(52) U.S. Cl. CPC C12Q 1/6806; C12Q 1/6804

(57) ABSTRACT

The invention relates to a single novel dry reagent composition for use in subsequent nucleic acid amplification, such as PCR (Polymerase Chain Reaction) and the isothermal LAMP (loop-mediated isothermal amplification), the composition including a nucleic acid amplification enzyme; a corresponding enzyme binder; a sugar-based stabiliser; deoxyNucleotide TriphosPhates; oligonucleotides; and a zwitterionic agent where a tris-based buffer is specifically excluded. The invention further relates to methods for producing said single dry reagent composition using air drying processes.

### FIELD OF INVENTION

The invention relates to dry, or substantially dry, nucleic acid amplification reagent compositions using zwitterion buffering and other additives to create a single biologically active thermostable amplification reagent. In particular, the invention facilitates methods of producing dry mixtures whilst avoiding the disadvantages associated with known/existing processes.

### BACKGROUND

A range of nucleic acid amplification methods has been developed in the art, such as PCR, LAMP and rolling circle amplification (RCA).

One of the most common and useful processes in molecular biology is nucleic acid amplification by PCR, a technique which allows the nucleic acid sequence at a specific region of a genome to be amplified by more than a million-fold. Parts of the nucleotide sequence adjacent to the areas to be amplified are used to hybridise synthetic nucleic acid oligonucleotides, one complimentary to each strand of the DNA helix. These oligonucleotides are used as primers to copy synthetically new regions of nucleic acid using, for example, a polymerase enzyme such as Taq polymerase, the four chemical bases adenosine, guanosine, cytidine and thymidine in the form of deoxy Nucleotide Triphosphates (dNTPs). Associated buffers and other salts needed for the reaction are also provided. Each cycle of the PCR requires a denaturation step to separate the two strands of the nucleic acid, an annealing step for specific hybridisation of complimentary nucleic acid sequences and extension step for the synthesis of DNA. Normally, up to 40 such cycles are required to allow sufficient amplification to be detected by one skilled in the art.

Other types of nucleic amplification technology use various combinations of oligonucleotides, enzymes, (d)NTPs and buffer reagents referred to above, although not all require thermal cycling steps (these are often known as isothermal amplification processes).

Nucleic acid amplification has wide application, such as the detection of genetic disease (e.g. cystic fibrosis), cancer, and infectious disease, both bacterial and viral (such as chlamydia and HIV).

Nucleic amplification can be utilised in conjunction with a variety of other techniques such as genomic sequencing and microarrays with further new techniques being anticipated as this area of biotechnology continues to evolve.

For amplification of target nucleic acids by PCR, critical components of the reaction mixture (PCR buffer) such as oligonucleotide primers, polymerase dNTPs, buffers and salts must be combined prior to adding the enzyme and the DNA before initiating the reaction. For reasons of performance and stability, providers of PCR assays for nucleic acid amplification normally provide the ingredients required in a portioned format, e.g. the oligonucleotides are supplied separately from the critical PCR components so that the end user has to combine PCR buffer, enzyme, oligonucleotides and sample, which can be cumbersome to add and mix small amounts of each component required for a given test sample and can result in errors in the reagent composition.

Alternatively, pre-mixing all reagents except sample DNA together can be achieved in liquid form and supplied to end-users frozen as the oligonucleotides, enzyme and the dNTPs will deteriorate at higher temperatures rendering the mix unusable after a relatively short period of time.

Another methodology of producing dry reaction components is to provide the vital ingredients compartmentalised and separate by means of lyophilisation, each component is then combined together and re-hydrated with water such as disclosed by patents WO2008/090340A1 and US2005/069898.

A well-known method of drying complete nucleic acid amplification mixtures is to combine the ingredients and lyophilise the reagents. For lyophilisation, the reagents are pre-mixed with a cryopreservative and then the whole mix lyophilised within a freeze dryer. The lyophilisation process relies on the understanding of the freezing and thermal properties of the reaction mixture, which can vary from mix to mix, depending on its exact composition. For instance, the presence of glycerol, an additive commonly used in the storage of the enzyme, will either alter the lyophilisation parameters, so they have to be re-optimised or indeed made the mix impossible to lyophilise. Lyophilisation is dependent on utilising a suitable vessel that can survive the extreme conditions in lyophilisation and supports the sublimation of water from the lyophilised material in a way that does not compromise the performance and stability of the lyophilised material. The lyophilisation equipment can be complex and expensive, consisting of heated and cooled shelves, vacuum chambers and condensers and therefore such methods remain challenging from a technical and commercial perspective. Lyophilised material also requires low humidity conditions post-lyophilisation such that lyophilised material has to be packed under dry inert gas or sealed whilst in a humidity controlled chamber; failure to stop humidity ingress in a lyophilised material will render the lyophilised product unstable over time and at temperatures elevated over ambient temperature.

There remains a requirement for a new solution that avoids some/all of the disadvantages arising in existing methods of producing stable reagent compositions for use in nucleic acid amplification.

Furthermore, a new reagent composition made in a new way, which addresses the practical challenges associated with the storage and transport of existing reagents, as acquired by an end user of DNA amplification processes, including PCR, sequencing and diagnosis of disease is highly desirable.

It is against this background that the current invention has arisen.

### SUMMARY OF THE INVENTION

In a first aspect the invention relates to a single air-dried reagent composition for nucleic acid amplification comprising: a nucleic acid amplification enzyme, a corresponding enzyme binder, a sugar-based stabiliser, deoxyNucleotideTriphosphates (dNTPs), oligonucleotides, salts and a zwitterion buffering agent for stabilisation of dNTPs within a complex mixture wherein the composition excludes tris-based buffers.

The invention particularly concerns a dry reagent composition prepared by air drying or evaporation for nucleic acid amplification comprising: at least one nucleic acid amplification enzyme; at least one corresponding enzyme binder; a sugar-based stabiliser; deoxy Nucleotide TriphosPhates (dNTPs) or Nucleoside Triphosphates (NTPs) or modified versions thereof; labelled or unlabeled oligonucleotides for nucleic acid amplification; and a zwitterionic agent, wherein the composition excludes at least Tris-based buffers and a nucleic acid template to be amplified.

The nucleic acid amplification enzyme may be any of one or more enzymes that are involved in the process of amplifying nucleic acids. Such enzymes include polymerases, DNA polymerases, transcriptases and other enzymes known in the art to be useful in the amplification of nucleic acids.

For example, DNA polymerase enzymes synthesise DNA complementary to a target sequence in the subsequent DNA replication. Such a polymerase generates new strands of DNA using a DNA template and primers (oligonucleotides). The enzymes may be a combination of different enzymes.

A corresponding enzyme binder or enzyme binder mix is required to be present in the reagent mix to block the activity of the enzyme in the reagent mix prior to the amplification process being initiated; it is believed this may contribute to the overall stability of the reagent composition. Such a component is adapted to interact with the enzyme in such a way that it temporarily blocks activity. The enzyme binding component may ultimately be disassociated rom the enzyme during the first step of nucleic acid amplification, for example by heat application.

For example, if DNA polymerase is used, the corresponding DNA polymerase binder, such as anti-Taq, prevents activity by the polymerase on the dTNPs whilst in the dry reagent mix. However, when the reagents are utilised in amplification the DNA polymerase is unblocked and its activity enabled, allowing the process, such as PCR, to continue as normal.

A sugar-based chemical is present in the mix to perform an additional stabilising function, such that the proteins therein are stabilised.

DeoxyNucleotideTriphosphates (dNTPs) or Nucleoside Triphosphates (NTPs) are typically comprised of a molecule containing a nucleoside bound to three phosphate groups which are building blocks for the new DNA strands and thus a key component in a one pot reagent mix useful in amplification. The nucleoside triphosphates containing deoxyribose take the prefix deoxy- in their names and small d- in their abbreviations for example: deoxyadenosine triphosphate (dATP), deoxyguanosine triphosphate (dGTP), deoxycytidine triphosphate (dCTP), deoxythymidine triphosphate (dTTP) and deoxyuridine triphosphate (dUTP).

dNTPs can be chemically modified with a variety of compounds to add functions to the nucleic acid amplified, for example to make it easier to detect the amplification in an assay. Chemically modified nucleotides can also be utilised to improve functionality of the nucleotides in an assay, for example modifications to make the nucleotides only available to enzymatic amplification once a certain temperature is reached, so called hot start dNTPs.

DNA polymerase can add a nucleotide only onto a pre-existing 3′-OH group, and as such it needs a primer to which it can add that first nucleotide. Oligonucleotides are short pieces of single-stranded DNA that are complementary to the target sequence and are thus provided as a component in the dried mix of the invention. DNA polymerase, when activated starts synthesizing new DNA from the end of the primer or oligonucleotide. Clearly the selection of the oligonucleotide makes it possible to delineate a specific region of template sequence required to be amplified in order to accumulate billions of copies (amplicons). The particular selection of the target sequence is therefore not limiting on the present invention.

A zwitterionic agent is provided in the reagent composition of the invention: this is a dipolar ion, that is, a neutral molecule with both positive and negative electrical charges. This component provides crucial buffering in the amplification mixture to provide a stabilising function to the reagent composition. The applicant believes such a component has an impact on the stability of the NTPs or dNTPs, which may therefore positively impact the technical functionality. Tris does not isomerise into zwitterionic species, however Tris-based buffers are traditionally used within nucleic acid amplification strategies, but the use of Tris-based buffers were discovered to be detrimental to stabilising a complete dry PCR reaction that only requires rehydration with sample for PCR. Tris-based buffers are therefore specifically excluded from the single reagent mix. Other solutions have been developed but necessarily use lyophilisation to avoid this issues e.g. Seise et al; Journal of Medical Microbiology (2013), 62, 1588-1591 have disclosed that evaporation drying a pre-PCR solution containing a tris-based PCR buffer (innuTAQ, Jenna analytic) all ingredients, and crucially dNTPs, does not yield a functioning reaction as the dNTPS are rendered inactive in such a composition and method. A compartmentalised delivery of system of separately drying dNTPs via polyolefin plastic and adding this to the other dry ingredients to make a functioning reaction was developed. The present applicants have however provided a new solution in the form of without the need for lyophilisation or compartmentalisation of key ingredients.

This single combination of dry pre-mixed reagents as disclosed herein provides a novel composition which is conveniently ready for when nucleic acid amplification is desired. Such a composition is stable at relatively high temperatures, such as at ambient temperatures (0° C. to 28 or 32° C.), than have been achieved by reagent compositions available in the art. The composition of the invention also provides a practical solution to the difficulties associated with the storage and transport of one-pot reagent compositions intended for use in amplification processes. Advantageously, the product reagent of the invention is maintained in a dry state for the physical storage and shipment of such material as compared to wet materials which would not be actively stable and be affected by evaporation, leakage, frothing and spillage.

In embodiments, the composition of the invention only excludes the sample/template nucleic acid necessary for amplification. That is, the dry mix composition is inclusive of all amplification reagents required, save the addition of the nucleic acid template for which replication is desired. The invention therefore comprises a single product avoiding the need for an end user to separately source reagents. A user seeking to undertake nucleic acid amplification is only required to add the template nucleic acid, such as DNA, to this composition.

Furthermore, the invention relates to a process for producing a single air dried pre-amplification reagent composition comprising: mixing together non-lyophilised components, including at least a nucleic acid amplification enzyme, a corresponding enzyme binder, a sugar-based stabiliser, Nucleoside TriPhosphates or deoxy Nucleotide Triphosphates or variations thereof, oligonucleotides and a zwitterionic agent together excluding any Tri-based buffer components, and subsequently drying the composition by air or evaporation methods and specifically excludes any lyophilisation step.

The air drying or evaporation process is essential as compared to rather than any lyophilisation process. The drying environment may be in the open air, preferably overnight. Alternatively the composition may be subjected to a warm environment in a cabinet, warm room or the like for example at 30 degrees C. Other typical embodiments within the remit of the invention include vacuum drying under ambient temperatures.

The method of the invention provides a convenient product which can be stored in a stable form, with no special requirements and no expensive or complex processes. The combination of components and process avoids the disadvantages associated with known methods, such as lyophilisation, which is required during production of the other dry reagent compositions described in the prior art

The applicants have therefore developed a useful method of producing and a composition of a long-term thermostable pre-mixed single reagent for use in nucleic acid amplification range of nucleic acid amplification methods, such as PCR, LAMP and rolling circle amplification (RCA).

The single pre-mixed dry reagent comprises all ingredients required for amplification, including but not limited to polymerase, nucleotides, magnesium chloride, buffering, salts and oligonucleotide primers and probes. The reagent becomes active on addition of a rehydrating nucleic acid which allows nucleic acid amplification and detection.

The method of producing the reagent utilises a convenient air-drying/evaporation system of drying that obviates the requirement to dry the mixture using expensive and time-consuming lyophilisation methods. The invention and method of producing the reagents delivers long-term thermostable pre-mixed reagents without the need for lyophilisation and the system is ideally suited to stabilising reagents for use in assays where stabilisation by lyophilisation is unsuitable.

Although lyophilising (freeze-drying) and drying by evaporation both result in dry or substantially dry reagents, prior to this invention there have been no disclosures that a lyophilised reagent retains the same activity when dried by other methods. The applicant has successfully been able to develop a product which utilises evaporation to produce a thermostable reagent composition. Although several attempts have been made it has been shown that prior to the present invention, e.g. by Seise et al; Journal of Medical Microbiology (2013), 62, 1588-1591, that reagents containing dNTPs within a complete nucleic acid amplification buffer does not work.

In examples, the at least one nucleic acid amplification enzyme is provided in a range from 0.01 to 2 units; and/or the at least one enzyme binder is provided in a range from 0.01 ug to 1 ug; and/or the sugar-based stabiliser is provided in a range from 0 to 10% v/v; and/or the Nucleoside Triphosphates or deoxyNucleotideTriphosPhates are each provided in a concentration range from 0.1 mM to 0.4 mM; and/or the oligonucleotides are provided in a concentration range from 0.001 mM to 101 mM; and/or the zwitterionic agent is provided in a concentration range from 10 mM to 100 mM.

The reagents are typically pre-mixed and dispensed in commercially desirable volumes before drying. In preferred embodiments, the dry composition of the invention is rehydrated with sample for use in amplification reactions in volumes of 5-20 uL and more preferably about 10ul, but not restricted to these volumes. Ultimately, the invention extends to a rehydrated composition in any volume, but especially to micro volumes thereof conveniently arranged for individual reaction use.

Any nucleic acid enzyme which has capability and suitability for nucleic acid amplification, and preferably PCR, may be used in preferred embodiments of the invention. Such an enzyme may be present in a range between 0.01 to 2 U (units), such as 0.75 U.

Such enzymes may be polymerases. For example, DNA polymerases generate new strands of DNA using a DNA template and primers and are thermostable or heat resistant.

In some embodiments of the invention, the polymerase enzyme is the commonly available “Taq” polymerase (Thermis aquaticus), which particularly suitable for PCR amplification techniques. In a preferred embodiment, Taq DNA polymerase is utilised. Alternatively, other DNA polymerases, such as Pfu (Pyrococcus furiosus), may be used due to higher fidelity when copying DNA.

The at least one enzyme maybe selected from but not limited to: polymerase, DNA polymerase, Taq polymerase, transcriptase and reverse transcriptase.

In other amplification techniques, one or more enzymes may be used. For example, LAMP is an isothermal nucleic acid amplification technique carried out with a series of alternating temperature cycles at a constant temperature of 60-65° C. (no thermal cycler). The target sequence is amplified at a constant temperature using either two or three sets of primers and the enzyme (e.g. polymerase) is/are selected with high strand displacement activity in addition to a replication activity.

### Enzyme Binder

The corresponding enzyme binder must be able to block the activity for the nucleic acid amplification enzyme and thus have a good affinity therefor. Typically the enzyme and corresponding enzyme binder will be provided in the composition in a molar ratio of approximately 1:1 to 1:1.05 but the invention is not restricted to this molar ratio.

The enzyme binding component maybe specific to the nucleic acid amplification enzyme comprised in the reagent composition of the invention. In particular, the enzyme binder may be specific for DNA polymerase or Taq polymerase and have a strong binding affinity therewith.

In some embodiments this component may be an antibody, antibody fragment, or synthetic molecules that serve such a function including aptamers, which bind polymerase.

The antibody maybe specific for taq polymerase. Taq binding molecules are usually known as ‘hot start’ reagents; as nucleic acid amplification cannot begin until the enzyme and its binder are dissociated, usually by heat. Hot start reagents are commonly used to improve the fidelity of amplification and remove amplification artefacts. Thus, in particularly preferred embodiments, Anti-taq mAB is provided as the enzyme binder.

In some embodiments the enzyme binding component is provided in the range of 0.01 to 1 ug per reaction, for example, 0.0825 μg for a given reaction in the composition of the invention.

### Sugar Stabiliser

The sugar-based compound is used to stabilise proteins. The sugar based compound may be provided in a range of 0 to 10% v/v, preferably about 8% v/v in the composition of the invention. In embodiments, any sugar based chemical that fulfil the stabilising function, such as Monosaccharides; Fructose, Galactose, Glucose, Rhamnose and Xylose, Disaccharides; Lactose, Maltose, Trehalose, Sucrose and Trisaccharides; Melezitose and Raffinose would be useful.

In some embodiments the sugar-based stabiliser is trehalose. In some embodiments this is provided as D-(+)-Trehalose dehydrate.

dNTPs/NTPs

In preferred embodiments the dNTP/NTP components may be modified or unmodified. In some embodiments, the NTP or dNTP components may each be provided in a working concentration range of 0.1 to 0.4 mM, preferably 0.125 mM.

As it concerns PCR, in some embodiments of the invention, modified dNTPs may be preferred for improved PCR performance. This may be principally due to their interaction with polypropylene, which is typically used in such reactions.

### Zwitterionic Agent

This dipolar agent is preferably in the form of an organic chemical buffer. This component may be provided in a concentration range of 10 to 100 mM, most preferably about 70 nM.

A preferred buffer of the invention is 4-(2-hydroxyethyl)-1-piperazineethanesulfonic acid or HEPES. Such a buffer is good at maintaining physiological pH despite changes in carbon dioxide concentration when compared to bicarbonate buffers. The dissociation of water decreases with falling temperature as compared to the dissociation constant (pK) of many other buffers, which does not change significantly with temperature. However, HEPES, like water, has a dissociation which decreases as the temperature decreases. The applicants have determined that HEPES is a particularly effective buffering agent for maintaining enzyme structure and function at variable temperatures in a dry formulation. Furthermore, HEPES is extremely effective when the enzyme used in the reagent mix is Taq polymerase and at the ranges provided above.

Other Zwitterionic agents useful in the invention may include some of the selection known as “Good's” buffers. These buffers display characteristics integral to biology and biochemistry. The characteristics associated include pKa value between 6.0 and 8.0, high solubility, non-toxic, limited effect on biochemical reactions, very low absorbance between 240 nm and 700 nm, enzymatic and hydrolytic stability, minimal changes due to temperature and concentration, limited effects due to ionic or salt composition of the solution, limited interaction with mineral cations, and limited permeability of biological membranes. However, some chemical buffers, such Tris, despite being in this category, are excluded from the remit of the invention as non-working embodiments. In particular Tris-based buffers and tricine-based buffers: 3-[(3-cholamidopropyl)dimethylammonio]-1-propanesulfonate (CHAPS), N-Cyclohexyl-2-aminoethanesulfonic acid (CHES) and N-cyclohexyl-2-hydroxyl-3-aminopropanesulfonic acid (CAPSO) are excluded.

### Oligonucleotides

In embodiments of the invention the oligonucleotides maybe provided in the concentration range of 0.001 mM to 1010 mM, preferably 0.01 to 0.5, with the precise determination of concentration being strongly dependent on the application. These components may be labelled.

### Other Components

In some utilisations, such as PCT the reagent mix of the invention may comprise additional components to optimise the application. For example, in some embodiments a further component, in the form of a divalent salt, such as a magnesium salt, may form part of the dry reagent composition.

In embodiments salts including, for example, Magnesium Chloride may be provided in a concentration range of 1 to 10 mM, more preferably 6 nM. The divalent magnesium ion (Mg++) is particularly relevant for PCR polymerase activity. In a preferred embodiment Magnesium Chloride may be utilised. Lower concentrations Mg++ will increase replication fidelity, while higher concentrations will introduce more mutations.

Other salts may include ammonium sulphate and or potassium chloride wherein one, or both is provided in a working concentration of up to 70 mM. For example, Ammonium Sulphate may be provided in a working concentration of at least as 2 mM and/or potassium chloride may be provided at 20 mM.

Other agents such as detergents, denaturants and colorants may further optimise the composition of the invention.

For example, denaturants (such as DMSO) may increase amplification specificity by destabilizing non-specific primer binding. In some embodiments therefore, the composition of the invention further comprises a denaturant.

Agents of use also include detergents which may help prevent the enzyme, such as polymerase, stick to itself or to the walls of the reaction tube. Therefore, in some embodiments a detergent may be provided in the composition of the invention. In embodiments the detergent is preferably provided in the range of 0 to 0.5% v/v and more preferably at 0.1% v/v. In particularly preferred embodiments the detergent is Triton X-100.

In some embodiments colourants, such as Patent Blue VF solution, may also be included in the composition of the invention. Such colourants may be provided at less than 0.001% v/v, preferably approximately at 0.00025% v/v.

### DETAILED DESCRIPTION

Table 1 exemplifies preferred embodiments of components and their working ranges for use in a working composition of the invention. Such example compositions are stable and therefore useful in nucleic acid amplification processes.

TABLE 1
Working Example Composition
Table 1 provides an example combination of the components of the composition of the invention.
Provided the essential components are present these may be available in any combination of the
above and in any working concentrations, without being limited there to, provided no tris-based
buffer is used.

| Dry reagent | | Example | Example working | |
|---|---|---|---|---|
| component | Range | Embodiment | concentration | Example supplier |
| Nucleic acid | 0.01 to 2 units | Taq Polymerase | 0.75 U | Qiagen, P7250-5000 |
| enzyme | | (Enzymatic taq B, | | |
| | | 5 U/μl) | | |
| Enzyme binder | 0.01 to 1 ug | Anti-taq mAB | 0.0825 μg per reaction | PCRBio CP004 |
| dNTPs | 0.1 to 0.4 mM | dNTPs | 0.125 mM each | Trilink/N-9506 |
| Sugar-based | 0 to 10% v/v | D-(+)-Trehalose | 8% v/v | Sigma T5251 |
| stabiliser | | dihydrate | | |
| Zwitterionic agent | 10 to 100 mM | HEPES, pH 8 | 70 mM | Sigma H0887-100 ml |
| Oligonucleotides | 0.001 mM to 10 mM | Oligonucleotides | 0.1-0.5 uM | various |

Optional components are also included, as indicated in parenthesis below, but are not essential.

| | | | | |
|---|---|---|---|---|
| Colourant (option) | <0.001% v/v | 0.05% Patent Blue VF | 0.00025% v/v | 198218 Sigma- |
| | | solution | | Aldrich |
| Detergent (option) | 0 to 0.5% v/v | Triton-X | 0.1% v/v | Sigma T8787 |
| Salt 2 component | 0 to 70 mM | Ammonium Sulphate | 2 mM | Sigma/A4418 |
| (option) | | | | |
| Salt 3 component | 0 to 70 mM | Potassium Chloride | 20 mM | Sigma/P9333 |
| (option) | | | | |
| Salt component | 1 to 10 mM | MgCl2 | 6 mM | Sigma M1028 |
| (option) | | | | |

### Comparative Testing

### Non-Working Examples

In this example, the effect of tris-based PCR buffers on dry compositions is provided in which six different preparations of dried PCR composition were made with varying concentrations of tris HCL ph8.3 as buffer. The PCR buffer composition is given as given in the table above where variant 1 is 240 mM Tris-HCl pH8.3, variant 2 is 200 mM, variant 3 is 160 mM, variant 4 is 120 mM, variant 5 is 80 mM and variant 6 is 40 mM. The PCR mixtures were completed by combining a 4× concentrated version and in a 4:1 ratio with a 10× concentration of oligonucleotides.

The resulting combination was dispensed in a volume of 5 μl in a standard white qPCR plate and submitted to air drying for 17 hours at 30° C. The resulting dried mixes were sealed with PCR caps and stored at different stability test conditions until use.

The oligonucleotides used in this experiment were a multiplexed set designed (reaction identity HPA-2ab.2) to detect the alleles encoding the Human Platelet Antigen 2a/b (HPA-2a & HPA-2b) using a consensus amplification flanking the 2a/2b polymorphism and dual labelled hydrolysis probes specific for the 482C>T nucleotide. The 482C position was detected with a probe labelled on 5′ with CalFluor Orange 560 fluorophore and with BHQ1 on the 3′ end, whereas the 482T position detected by another probe 5′ labelled with FAM and BHQ1 quenched at the 3′ end. Oligonucleotides specific for a consensus region of the human growth hormone (HGH) were included as an ‘internal amplification control’ (IAC). The IAC amplification was detected using a probe labelled with CalFlour Red 610 and quenched with BHQ2 at the 3′ end.

The dried mixes were subjected to storage at 1 or 2 weeks at both 32° C. and 37° C. and compared to immediate (Time point 0) conditions.

The dried PCR mixes were tested at each timepoint in duplicate by rehydrating the dried samples with 10ul of 10 ng/μl reference DNA sample (heterozygous for HPA-2a/b). Successful PCR is measured by the final fluorescence measurement (FF) and the crossing point (Cp) when the amplification is deemed to have started.

PCR was performed at various timepoints using a Bio-Rad CFX Touch qPCR instrument and data was reported, exported and tabulated as below.

TABLE 2
Stability data on Tris-based air dried compositions in PCR

| | Tris | | | | | | | | | | |
|---|---|---|---|---|---|---|---|---|---|---|---|
| | HCL | No storage | | 32° C. storage | | | | 37° C. storage | | | |
| Reaction | pH8.3 | FF | Cp | FF | Cp | FF | Cp | FF | Cp | FF | Cp |
| Id | conc | TP0 | | TP1 (1 week) | | TP2 (2 week) | | TP1 (1 week) | | TP2 (2 week) | |
| | | CalFluor Red 610 | | | | | | | | | |
| HPA- | 240 mM | 855,244 | 25.99 | 17,863 | 0.00 | 2,321 | 0.00 | 5,123 | 0.00 | 1,241 | 0.00 |
| 2ab.2 | | | | | | | | | | | |
| HPA- | 240 mM | 746,407 | 26.03 | 15,773 | 0.00 | 6,512 | 0.00 | 2,486 | 0.00 | 40,727 | 0.00 |
| 2ab.2 | | | | | | | | | | | |
| HPA- | 200 mM | 858,225 | 25.55 | 43,819 | 0.00 | 7,001 | 0.00 | 12,561 | 0.00 | 3,317 | 0.00 |
| 2ab.2 | | | | | | | | | | | |
| HPA- | 200 mM | 842,591 | 25.70 | 48,374 | 0.00 | 2,409 | 0.00 | 2,470 | 0.00 | 1,320 | 0.00 |
| 2ab.2 | | | | | | | | | | | |
| HPA- | 160 mM | 852,360 | 25.63 | 159,211 | 34.44 | 3,538 | 0.00 | 10,051 | 0.00 | 2,210 | 0.00 |
| 2ab.2 | | | | | | | | | | | |
| HPA- | 160 mM | 917,672 | 25.52 | 155,626 | 35.17 | 7,048 | 0.00 | 14,267 | 0.00 | 1,171 | 0.00 |
| 2ab.2 | | | | | | | | | | | |
| HPA- | 120 mM | 874,335 | 25.73 | 442,879 | 28.11 | 23,658 | 0.00 | 21,775 | 0.00 | 2,442 | 0.00 |
| 2ab.2 | | | | | | | | | | | |
| HPA- | 120 mM | 840,110 | 25.53 | 362,657 | 28.84 | 19,570 | 0.00 | 22,672 | 0.00 | 510 | 0.00 |
| 2ab.2 | | | | | | | | | | | |
| HPA- | 80 mM | 652,546 | 25.46 | 404,273 | 26.49 | 183,206 | 33.04 | 195,822 | 30.82 | 6,435 | 0.00 |
| 2ab.2 | | | | | | | | | | | |
| HPA- | 80 mM | 780,706 | 25.74 | 471,135 | 26.46 | 449,766 | 28.00 | 321,308 | 28.44 | 33,658 | 0.00 |
| 2ab.2 | | | | | | | | | | | |
| HPA- | 40 mM | 484,665 | 26.36 | 396,065 | 27.26 | 116,940 | 38.98 | 331,813 | 26.18 | 1,519 | 0.00 |
| 2ab.2 | | | | | | | | | | | |
| HPA- | 40 mM | 507,995 | 26.17 | 440,107 | 26.91 | 167,117 | 30.66 | 333,124 | 26.17 | 13,638 | 0.00 |
| 2ab.2 | | | | | | | | | | | |
| | | CalFluor Orange 560 | | | | | | | | | |
| HPA- | 240 mM | 574,018 | 28.49 | 285,632 | 34.42 | 8,485 | 0.00 | 5,700 | 0.00 | 2,416 | 0.00 |
| 2ab.2 | | | | | | | | | | | |
| HPA- | 240 mM | 497,642 | 28.39 | 280,499 | 35.88 | 25,880 | 0.00 | 2,982 | 0.00 | 6,450 | 0.00 |
| 2ab.2 | | | | | | | | | | | |
| HPA- | 200 mM | 521,620 | 27.78 | 325,336 | 32.11 | 35,537 | 0.00 | 3,735 | 0.00 | −228 | 0.00 |
| 2ab.2 | | | | | | | | | | | |
| HPA- | 200 mM | 558,661 | 27.98 | 354,844 | 31.46 | 2,251 | 0.00 | 3,319 | 0.00 | 767 | 0.00 |
| 2ab.2 | | | | | | | | | | | |
| HPA- | 160 mM | 510,763 | 27.92 | 357,024 | 28.94 | 14,236 | 0.00 | 123,103 | 40.17 | 1,482 | 0.00 |
| 2ab.2 | | | | | | | | | | | |
| HPA- | 160 mM | 498,380 | 27.83 | 388,371 | 29.08 | 49,351 | 0.00 | 126,058 | 38.99 | 207 | 0.00 |
| 2ab.2 | | | | | | | | | | | |
| HPA- | 120 mM | 428,867 | 27.90 | 362,364 | 27.54 | 164,961 | 34.91 | 234,497 | 31.79 | 3,336 | 0.00 |
| 2ab.2 | | | | | | | | | | | |
| HPA- | 120 mM | 443,102 | 27.35 | 362,101 | 27.93 | 165,665 | 34.35 | 249,253 | 31.05 | 4,111 | 0.00 |
| 2ab.2 | | | | | | | | | | | |
| HPA- | 80 mM | 314,716 | 27.40 | 333,146 | 27.82 | 312,552 | 29.03 | 364,106 | 26.78 | 62,106 | 44.09 |
| 2ab.2 | | | | | | | | | | | |
| HPA- | 80 mM | 359,741 | 27.87 | 378,057 | 27.83 | 326,262 | 28.78 | 359,394 | 27.25 | 174,458 | 34.40 |
| 2ab.2 | | | | | | | | | | | |
| HPA- | 40 mM | 177,927 | 28.98 | 240,426 | 29.60 | 124,410 | 31.25 | 289,342 | 27.99 | 60,020 | 44.04 |
| 2ab.2 | | | | | | | | | | | |
| HPA- | 40 mM | 192,608 | 28.61 | 289,458 | 28.96 | 153,513 | 30.83 | 306,511 | 27.73 | 89,670 | 35.98 |
| 2ab.2 | | | | | | | | | | | |
| | | FAM | | | | | | | | | |
| HPA- | 240 mM | 1,343,596 | 28.52 | 1,011,692 | 33.61 | 225,478 | 39.33 | 47,507 | 0.00 | 112,079 | 0.00 |
| 2ab.2 | | | | | | | | | | | |
| HPA- | 240 mM | 1,103,278 | 29.02 | 1,371,969 | 34.30 | 59,805 | 0.00 | 19,816 | 0.00 | 106,666 | 0.00 |
| 2ab.2 | | | | | | | | | | | |
| HPA- | 200 mM | 1,318,780 | 27.90 | 1,154,808 | 31.28 | 236,601 | 38.54 | 19,842 | 0.00 | 9,873 | 0.00 |
| 2ab.2 | | | | | | | | | | | |
| HPA- | 200 mM | 1,229,919 | 28.05 | 1,128,279 | 31.44 | 13,896 | 0.00 | 16,314 | 0.00 | 12,769 | 0.00 |
| 2ab.2 | | | | | | | | | | | |
| HPA- | 160 mM | 1,272,638 | 27.75 | 1,209,810 | 28.67 | 163,749 | 47.63 | 982,272 | 36.27 | 17,774 | 0.00 |
| 2ab.2 | | | | | | | | | | | |
| HPA- | 160 mM | 1,334,130 | 27.99 | 1,313,123 | 28.77 | 443,204 | 41.08 | 942,343 | 36.02 | 12,002 | 0.00 |
| 2ab.2 | | | | | | | | | | | |
| HPA- | 120 mM | 1,243,945 | 27.71 | 1,055,108 | 27.58 | 931,887 | 32.64 | 1,090,081 | 30.70 | 17,671 | 0.00 |
| 2ab.2 | | | | | | | | | | | |
| HPA- | 120 mM | 1,206,441 | 27.85 | 1,106,790 | 28.24 | 946,335 | 32.67 | 1,128,047 | 30.17 | 54,342 | 0.00 |
| 2ab.2 | | | | | | | | | | | |
| HPA- | 80 mM | 1,164,727 | 26.30 | 1,239,714 | 27.49 | 1,154,421 | 28.95 | 1,168,007 | 27.35 | 625,679 | 34.67 |
| 2ab.2 | | | | | | | | | | | |
| HPA- | 80 mM | 1,040,246 | 27.73 | 1,272,979 | 28.03 | 891,815 | 29.30 | 1,060,398 | 27.56 | 903,965 | 33.06 |
| 2ab.2 | | | | | | | | | | | |
| HPA- | 40 mM | 638,441 | 29.02 | 883,271 | 30.00 | 700,234 | 29.22 | 1,055,898 | 27.95 | 541,851 | 29.27 |
| 2ab.2 | | | | | | | | | | | |
| HPA- | 40 mM | 665,639 | 28.50 | 1,038,919 | 29.47 | 852,584 | 28.95 | 1,061,526 | 28.18 | 664,476 | 29.05 |
| 2ab.2 | | | | | | | | | | | |

The data in Table 2 shows that the concentration of tris at time point zero (just after drying) does not have any influence on amplification as all three fluorescent channels show similar Cp values although lower levels of tris are detrimental to the fluorescence. It can be seen that at 32° C. storage of 1 week the Cp values are starting to drift to larger numbers showing a loss of activity and the fluorescence values are diminishing showing less efficacy in amplification.

After two weeks at 32° C. the reactions at higher concentrations are non-functional.

The pattern is repeated but exaggerated in the 37° C. tests where it can be seen that at 2 weeks at 37° C. the dried reaction containing only tris has completely failed in the red fluorescence, is very poor in the orange fluorescence and just about working only at low concentrations in the FAM channel.

### Working Examples

The experiment (with the same conditions as above) was then repeated, this time replacing the tris buffers with HEPES pH 8.0 at varying concentrations, in accordance with an embodiment of the invention.

The HEPES containing buffers were dried as per the methods previously described and the results shown in Table 3

TABLE 3
data on 3-week stability using HEPES in air dried composition

| HEPES | TP0 | | TP1 28 c | | TP1 37 c | | TP2 28 c | | TP2 37 c | | TP3 28 c | | TP3 37 c | |
|---|---|---|---|---|---|---|---|---|---|---|---|---|---|---|
| pH8.0 | FF | CP | FF | CP | FF | CP | FF | CP | FF | CP | FF | CP | FF | CP |
| | CalFlour RED610 | | | | | | | | | | | | | |
| 20 | 892,091 | 25.79 | 982,917 | 25.72 | 729,868 | 26.37 | 746,778 | 26.08 | 5,411 | #DIV/0! | 681,876 | 26.73 | 7,698 | #DIV/0! |
| mM | | | | | | | | | | | | | | |
| 30 | 1,008,673 | 25.73 | 1,108,360 | 25.53 | 971,687 | 26.03 | 903,910 | 25.96 | 533,877 | 27.49 | 796,482 | 26.27 | 225,308 | 32.85 |
| mM | | | | | | | | | | | | | | |
| 40 | 1,074,817 | 25.63 | 1,008,663 | 25.70 | 918,775 | 25.89 | 1,010,065 | 25.76 | 668,233 | 27.01 | 880,033 | 26.09 | 400,279 | 27.20 |
| mM | | | | | | | | | | | | | | |
| 50 | 1,008,634 | 25.65 | 1,030,270 | 25.72 | 900,183 | 25.96 | 938,229 | 25.96 | 648,531 | 26.42 | 784,684 | 26.21 | 381,381 | 26.97 |
| mM | | | | | | | | | | | | | | |
| 60 | 1,212,936 | 25.24 | 1,212,725 | 25.49 | 871,449 | 25.85 | 1,086,757 | 25.39 | 830,128 | 25.75 | 914,503 | 25.82 | 152,109 | 28.77 |
| mM | | | | | | | | | | | | | | |
| 70 | 1,278,079 | 25.31 | 1,170,598 | 25.65 | 1,064,040 | 25.70 | 1,126,608 | 25.44 | 985,246 | 25.72 | 938,929 | 26.05 | 701,556 | 26.32 |
| mM | | | | | | | | | | | | | | |
| 80 | 1,219,190 | 25.40 | 1,070,810 | 25.72 | 1,061,808 | 25.86 | 1,042,284 | 25.50 | 935,677 | 26.55 | 1,056,785 | 26.07 | 618,266 | 27.44 |
| mM | | | | | | | | | | | | | | |
| 90 | 1,167,274 | 25.44 | 1,097,234 | 26.03 | 1,004,293 | 26.06 | 1,098,709 | 25.47 | 679,583 | 26.46 | 972,910 | 26.16 | 325,961 | 28.52 |
| mM | | | | | | | | | | | | | | |
| | Caflour Orange 560 | | | | | | | | | | | | | |
| 20 | 447,228 | 26.94 | 530,149 | 26.86 | 439,731 | 27.68 | 478,255 | 26.72 | 21,888 | #DIV/0! | 455,122 | 27.22 | 25,099 | #DIV/0! |
| mM | | | | | | | | | | | | | | |
| 30 | 550,317 | 26.80 | 568,917 | 27.10 | 525,460 | 27.70 | 549,970 | 26.55 | 407,173 | 28.03 | 482,825 | 27.15 | 239,941 | 29.84 |
| mM | | | | | | | | | | | | | | |
| 40 | 604,078 | 27.19 | 555,716 | 27.20 | 516,609 | 27.58 | 601,661 | 26.94 | 444,838 | 27.85 | 528,893 | 27.47 | 356,236 | 27.90 |
| mM | | | | | | | | | | | | | | |
| 50 | 589,227 | 27.11 | 557,454 | 27.85 | 515,372 | 27.31 | 562,933 | 27.36 | 422,780 | 27.70 | 490,287 | 27.32 | 346,233 | 27.24 |
| mM | | | | | | | | | | | | | | |
| 60 | 636,969 | 26.51 | 689,121 | 26.72 | 539,079 | 27.06 | 637,489 | 26.32 | 569,438 | 26.85 | 529,404 | 26.60 | 204,088 | 30.11 |
| mM | | | | | | | | | | | | | | |
| 70 | 738,288 | 26.51 | 667,991 | 27.27 | 636,669 | 27.23 | 667,699 | 26.41 | 635,250 | 27.14 | 574,773 | 27.18 | 515,480 | 26.83 |
| mM | | | | | | | | | | | | | | |
| 80 | 711,484 | 27.30 | 594,445 | 27.74 | 614,528 | 27.68 | 619,347 | 26.89 | 552,562 | 28.12 | 614,056 | 27.46 | 524,090 | 26.64 |
| mM | | | | | | | | | | | | | | |
| 90 | 696,240 | 27.31 | 664,984 | 26.86 | 579,623 | 27.68 | 633,991 | 27.31 | 410,055 | 28.10 | 614,821 | 26.61 | 318,171 | 28.03 |
| mM | | | | | | | | | | | | | | |
| | FAM | | | | | | | | | | | | | |
| 20 | 1,374,242 | 26.54 | 1,391,435 | 26.95 | 1,330,491 | 28.02 | 1,379,889 | 26.11 | 30,857 | #DIV/0! | 1,363,067 | 27.07 | 312,227 | 34.89 |
| mM | | | | | | | | | | | | | | |
| 30 | 1,550,810 | 26.01 | 1,479,267 | 27.30 | 1,330,151 | 28.37 | 1,555,453 | 25.38 | 1,251,725 | 28.20 | 1,389,686 | 27.14 | 1,192,865 | 29.26 |
| mM | | | | | | | | | | | | | | |
| 40 | 1,447,142 | 27.84 | 1,371,329 | 27.61 | 1,306,909 | 27.76 | 1,502,855 | 26.77 | 1,131,914 | 28.66 | 1,293,229 | 28.02 | 1,420,533 | 28.24 |
| mM | | | | | | | | | | | | | | |
| 50 | 1,486,067 | 27.03 | 1,441,244 | 26.63 | 1,331,521 | 27.59 | 1,439,182 | 26.72 | 1,194,653 | 28.03 | 1,296,000 | 26.65 | 1,573,202 | 26.67 |
| mM | | | | | | | | | | | | | | |
| 60 | 1,656,613 | 25.92 | 1,597,425 | 27.10 | 1,437,913 | 27.02 | 1,559,157 | 25.92 | 1,429,090 | 27.30 | 1,416,478 | 27.05 | 1,123,832 | 29.32 |
| mM | | | | | | | | | | | | | | |
| 70 | 1,605,305 | 27.40 | 1,392,401 | 28.25 | 1,376,084 | 28.12 | 1,566,966 | 25.89 | 1,453,600 | 27.94 | 1,285,866 | 28.03 | 1,818,891 | 26.21 |
| mM | | | | | | | | | | | | | | |
| 80 | 1,428,624 | 28.42 | 1,296,857 | 28.35 | 1,341,319 | 28.41 | 1,404,135 | 27.15 | 1,312,113 | 28.78 | 1,369,914 | 28.40 | 1,577,736 | 27.94 |
| mM | | | | | | | | | | | | | | |
| 90 | 1,481,640 | 27.22 | 1,350,244 | 27.85 | 1,291,958 | 28.59 | 1,400,952 | 27.84 | 1,055,228 | 28.77 | 1,374,016 | 27.22 | 1,273,325 | 28.29 |
| nM | | | | | | | | | | | | | | |

At compositions above 40 mM both final fluorescence values (FF) and Cp values were stable at 37° C. for at least 3 weeks compared to failure at two weeks for the Tris-based versions shown in Table 2.

### Long-Term Stability Study

A composition as per Table 1 in accordance with embodiments of the invention was formulated for long term stability studies at three different temperatures: −20° C., 4° C. and 32° C., shown in Tables 4a, 4b and 4c, respectively below.

Two different real time PCR primer and probe sets were utilised and given the reaction identities HPA-1ab and BG65. Reactions were prepared as previously described and the PCR plate strips were sealed with PCR caps and sealed in a foil pouch with a desiccant pouch to prevent light and moisture ingress. The real time 3-plex PCR reactions were both utilising the same HGH IAC oligos using CalFluor Red610 fluorescence as previously described, and the two allele channels utilised were CalFluor Orange 560 and FAM.

A ‘day zero test’ was used to establish the baseline for the two real time PCR reactions with the two DNA samples utilised throughout the experiment.

The reactions were considered positive if the following conditions were met:

| | |
|---|---|
| Red channel +ve if | FF > 1,000,000 RFU, & Cp > 25 < 34 |
| Orange channel +ve if | FF > 800,000 RFU, & Cp > 25 < 33 |
| FAM channel +ve if | FF > 1,000,000 RFU, & Cp > 25 < 34 |

The study illustrates that those example embodiments, using dry reaction components as disclosed herein in accordance with the invention remained active after 1 year at the elevated temperature of 32° C. This data compares favourably to tris-based reaction examples that are not stable after 2 weeks at 32° C.

TABLE 4
1 year stability data
Table 4a

| | | | | | Stored −20° C. in foil pouch with desiccant | | | | | | | | | |
|---|---|---|---|---|---|---|---|---|---|---|---|---|---|---|
| | | | | | −20° C., 1 | | −20° C., 2 | | −20° C., 4 | | −20° C., 8 | | −20° C., 12 | |
| | | | day zero | | month | | months | | months | | months | | months | |
| | | | Plate 1 | | Plate 61 | | Plate 70 | | Plate 79 | | Plate 88 | | Plate 97 | |
| Reac- | | | R610 | | R610 | | R610 | | R610 | | R610 | | R610 | |
| tion | D | Ex- | Mean | Mean | Mean | Mean | Mean | Mean | Mean | Mean | Mean | Mean | Mean | Mean |
| ID | NA | pected | FF | CP | FF | CP | FF | CP | FF | CP | FF | CP | FF | CP |
| HPA- | DN | + | 4,274,163 | 27.50 | 4,465,486 | 27.35 | 4,277,193 | 27.25 | 4,635,319 | 26.76 | 4,307,561 | 26.19 | 4,083,307 | 27.46 |
| 1ab | A1 | | | | | | | | | | | | | |
| HPA- | DN | + | 3,920,229 | 27.93 | 4,136,895 | 27.66 | 4,072,936 | 27.33 | 4,516,147 | 27.66 | 4,108,928 | 26.22 | 3,792,774 | 27.56 |
| 1ab | A2 | | | | | | | | | | | | | |
| BG65 | DN | + | 3,806,896 | 27.67 | 4,158,943 | 27.36 | 4,019,397 | 27.25 | 4,291,091 | 27.09 | 3,997,458 | 26.21 | 4,041,178 | 27.59 |
| | A1 | | | | | | | | | | | | | |
| BG65 | DN | + | 4,047,448 | 27.89 | 4,702,533 | 27.26 | 4,225,402 | 27.33 | 4,423,207 | 27.93 | 4,417,546 | 25.99 | 4,200,765 | 27.48 |
| | A2 | | | | | | | | | | | | | |

Table 4a

| | | | Plate 1 | | Plate 61 | | Plate 70 | | Plate 79 | | Plate 88 | | Plate 97 | |
|---|---|---|---|---|---|---|---|---|---|---|---|---|---|---|
| Reac- | | | O560 | | O560 | | O560 | | O560 | | O560 | | O560 | |
| tion | D | Ex- | Mean | Mean | Mean | Mean | Mean | Mean | Mean | Mean | Mean | Mean | Mean | Mean |
| ID | NA | pected | FF | CP | FF | CP | FF | CP | FF | CP | FF | CP | FF | CP |
| HPA- | DN | + | 1,855,897 | 30.75 | 1,947,722 | 30.58 | 1,860,973 | 30.46 | 2,052,017 | 30.03 | 1,927,184 | 29.91 | 1,834,431 | 30.69 |
| 1ab | A1 | | | | | | | | | | | | | |
| BG65 | DN | + | 5,960,056 | 30.11 | 6,697,150 | 29.68 | 6,256,776 | 29.36 | 6,700,824 | 30.00 | 6,504,509 | 27.54 | 6,414,525 | 29.71 |
| | A2 | | | | | | | | | | | | | |
| HPA- | DN | − | 198,758 | 0.00 | 215,571 | 0.00 | 285,987 | 0.00 | 299,193 | 0.00 | 190,562 | 0.00 | 179,967 | 0.00 |
| 1ab | A1 | | | | | | | | | | | | | |
| BG65 | DN | − | 5,232,042 | 36.27 | 5,994,140 | 35.19 | 5,669,089 | 35.50 | 5,811,295 | 35.98 | 5,967,236 | 33.72 | 5,849,151 | 35.76 |
| | A2 | | | | | | | | | | | | | |

Table 4a

| | | | Plate 1 | | Plate 61 | | Plate 70 | | Plate 79 | | Plate 88 | | Plate 97 | |
|---|---|---|---|---|---|---|---|---|---|---|---|---|---|---|
| Reac- | | | FAM | | FAM | | FAM | | FAM | | FAM | | FAM | |
| tion | D | Ex- | Mean | Mean | Mean | Mean | Mean | Mean | Mean | Mean | Mean | Mean | Mean | Mean |
| ID | NA | pected | FF | CP | FF | CP | FF | CP | FF | CP | FF | CP | FF | CP |
| HPA- | DN | + | 11,330,278 | 27.98 | 12,087,051 | 27.83 | 11,846,478 | 27.66 | 12,471,076 | 27.33 | 11,348,168 | 27.15 | 10,947,773 | 27.96 |
| 1ab | A1 | | | | | | | | | | | | | |
| HPA- | DN | + | 14,193,985 | 27.59 | 15,431,130 | 27.47 | 15,079,926 | 27.10 | 16,712,622 | 27.56 | 14,716,029 | 26.57 | 14,049,945 | 27.30 |
| 1ab | A2 | | | | | | | | | | | | | |
| BG65 | DN | − | −342,350 | 0.00 | −392,106 | 0.00 | −302,871 | 0.00 | −400,031 | 0.00 | −441,206 | 0.00 | −476,491 | 0.00 |
| | A1 | | | | | | | | | | | | | |
| BG65 | DN | − | −362,061 | 0.00 | −388,242 | 0.00 | −388,176 | 0.00 | −401,190 | 0.00 | −465,862 | 0.00 | −467,381 | 0.00 |
| | A2 | | | | | | | | | | | | | |

Table 4b

| | | | Stored 4° C. in foil pouch with desiccant | | | | | | | | | |
|---|---|---|---|---|---|---|---|---|---|---|---|---|
| | | | 4° C., 1 month | | 4° C., 2 months | | 4° C., 4 months | | 4° C., 8 months | | 4° C., 12 months | |
| | | | Plate 62 | | Plate 71 | | Plate 80 | | Plate 89 | | Plate 98 | |
| Reac- | | Ex- | R610 | | R610 | | R610 | | R610 | | R610 | |
| tion | D | pected | Mean | Mean | Mean | Mean | Mean | Mean | Mean | Mean | Mean | Mean |
| ID | NA | result | FF | CP | FF | CP | FF | CP | FF | CP | FF | CP |
| HPA- | DN | + | 4,655,499 | 27.20 | 4,513,128 | 26.14 | 4,039,185 | 27.22 | 4,082,121 | 27.39 | 3,835,899 | 27.53 |
| 1ab | A1 | | | | | | | | | | | |
| HPA- | DN | + | 4,262,928 | 27.49 | 4,113,765 | 26.27 | 3,830,204 | 27.36 | 3,912,511 | 27.93 | 3,681,906 | 27.53 |
| 1ab | A2 | | | | | | | | | | | |
| BG65 | DN | + | 4,100,826 | 27.68 | 4,292,196 | 25.91 | 3,817,734 | 27.26 | 3,890,431 | 28.15 | 3,795,536 | 27.62 |
| | A1 | | | | | | | | | | | |
| BG65 | DN | + | 4,579,944 | 27.53 | 4,528,343 | 25.89 | 3,905,266 | 27.46 | 3,863,082 | 28.29 | 3,954,394 | 27.53 |
| | A2 | | | | | | | | | | | |

Table 4b

| | | | Plate 62 | | Plate 71 | | Plate 80 | | Plate 89 | | Plate 98 | |
|---|---|---|---|---|---|---|---|---|---|---|---|---|
| Reac- | | Ex- | O560 | | O560 | | O560 | | O560 | | O560 | |
| tion | D | pected | Mean | Mean | Mean | Mean | Mean | Mean | Mean | Mean | Mean | Mean |
| ID | NA | result | FF | CP | FF | CP | FF | CP | FF | CP | FF | CP |
| HPA- | DN | + | 2,027,601 | 30.40 | 1,955,508 | 29.64 | 1,743,712 | 30.63 | 1,835,866 | 30.68 | 1,679,630 | 30.94 |
| 1ab | A1 | | | | | | | | | | | |
| BG65 | DN | + | 6,729,158 | 29.55 | 6,426,464 | 28.24 | 5,907,745 | 29.31 | 6,193,300 | 29.66 | 6,271,353 | 28.87 |
| | A2 | | | | | | | | | | | |
| HPA- | DN | − | 223,489 | 0.00 | 256,879 | 0.00 | 250,575 | 0.00 | 153,702 | 0.00 | 163,367 | 0.00 |
| 1ab | A1 | | | | | | | | | | | |
| BG65 | DN | − | 6,000,782 | 35.32 | 5,872,005 | 34.63 | 5,263,808 | 35.55 | 5,691,067 | 35.57 | 5,806,485 | 34.52 |
| | A2 | | | | | | | | | | | |

Table 4b

| | | | Plate 62 | | Plate 71 | | Plate 80 | | Plate 89 | | Plate 98 | |
|---|---|---|---|---|---|---|---|---|---|---|---|---|
| Reac- | | Ex- | FAM | | FAM | | FAM | | FAM | | FAM | |
| tion | D | pected | Mean | Mean | Mean | Mean | Mean | Mean | Mean | Mean | Mean | Mean |
| ID | NA | result | FF | CP | FF | CP | FF | CP | FF | CP | FF | CP |
| HPA- | DN | + | 12,565,316 | 27.68 | 11,903,427 | 27.03 | 10,735,200 | 27.77 | 10,998,578 | 27.86 | 10,431,208 | 28.01 |
| 1ab | A1 | | | | | | | | | | | |
| HPA- | DN | + | 15,774,975 | 27.32 | 14,809,750 | 26.58 | 14,135,356 | 27.15 | 14,226,396 | 27.76 | 13,684,465 | 27.41 |
| 1ab | A2 | | | | | | | | | | | |
| BG65 | DN | − | −390,599 | 0.00 | −355,787 | 0.00 | −361,781 | 0.00 | −429,394 | 0.00 | −467,773 | 0.00 |
| | A1 | | | | | | | | | | | |
| BG65 | DN | − | −412,632 | 0.00 | −361,592 | 0.00 | −344,490 | 0.00 | −441,525 | 0.00 | −493,004 | 0.00 |
| | A2 | | | | | | | | | | | |

Table 4c

| | | | Stored 32° C. in foil pouch with desiccant | | | | | | | | | |
|---|---|---|---|---|---|---|---|---|---|---|---|---|
| | | | 32° C., 1 month | | 32° C., 2 months | | 32° C., 4 months | | 32° C., 8 months | | 32° C., 12 months | |
| | | | Plate 63 | | Plate 72 | | Plate 81 | | Plate 90 | | Plate 99 | |
| Reac- | | Ex- | R610 | | R610 | | R610 | | R610 | | R610 | |
| tion | D | pected | Mean | Mean | Mean | Mean | Mean | Mean | Mean | Mean | Mean | Mean |
| ID | NA | result | FF | CP | FF | CP | FF | CP | FF | CP | FF | CP |
| HPA- | DN | + | 4,425,588 | 27.24 | 4,579,700 | 27.09 | 4,035,520 | 26.18 | 3,989,009 | 26.25 | 3,453,396 | 27.11 |
| 1ab | A1 | | | | | | | | | | | |
| HPA- | DN | + | 4,422,687 | 27.21 | 3,558,889 | 27.45 | 3,867,236 | 36.38 | 3,936,280 | 26.29 | 3,554,085 | 27.62 |
| 1ab | A2 | | | | | | | | | | | |
| BG65 | DN | + | 4,145,554 | 27.29 | 3,738,079 | 27.39 | 3,898,030 | 26.17 | 4,009,788 | 26.15 | 3,742,340 | 27.44 |
| | A1 | | | | | | | | | | | |
| BG65 | DN | + | 4,504,940 | 27.23 | 4,289,849 | 27.48 | 4,067,059 | 26.27 | 4,759,142 | 25.48 | 3,830,815 | 27.55 |
| | A2 | | | | | | | | | | | |

Table 4c

| | | | Plate 63 | | Plate 72 | | Plate 81 | | Plate 90 | | Plate 99 | |
|---|---|---|---|---|---|---|---|---|---|---|---|---|
| Reac- | | Ex- | O560 | | O560 | | O560 | | O560 | | O560 | |
| tion | D | pected | Mean | Mean | Mean | Mean | Mean | Mean | Mean | Mean | Mean | Mean |
| ID | NA | result | FF | CP | FF | CP | FF | CP | FF | CP | FF | CP |
| HPA- | DN | + | 1,906,248 | 30.53 | 1,932,223 | 30.52 | 1,657,414 | 30.17 | 1,703,615 | 30.19 | 1,356,415 | 31.18 |
| 1ab | A1 | | | | | | | | | | | |
| BG65 | DN | + | 6,652,584 | 29.48 | 6,148,614 | 29.78 | 5,835,810 | 28.29 | 6,034,462 | 27.98 | 5,587,700 | 30.62 |
| | A2 | | | | | | | | | | | |
| HPA- | DN | − | 210,699 | 0.00 | 213,110 | 0.00 | 200,827 | 0.00 | 162,424 | 0.00 | 126,578 | 0.00 |
| 1ab | A1 | | | | | | | | | | | |
| BG65 | DN | − | 5,916,969 | 35.79 | 3,944,496 | 36.38 | 5,469,641 | 34.81 | 5,836,179 | 34.73 | 4,984,385 | 36.99 |
| | A2 | | | | | | | | | | | |

Table 4c

| | | | Plate 63 | | Plate 72 | | Plate 81 | | Plate 90 | | Plate 99 | |
|---|---|---|---|---|---|---|---|---|---|---|---|---|
| Reac- | | Ex- | FAM | | FAM | | FAM | | FAM | | FAM | |
| tion | D | pected | Mean | Mean | Mean | Mean | Mean | Mean | Mean | Mean | Mean | Mean |
| ID | NA | result | FF | CP | FF | CP | FF | CP | FF | CP | FF | CP |
| HPA- | DN | + | 11,992,451 | 27.69 | 11,407,593 | 27.66 | 10,455,389 | 27.15 | 10,341,138 | 27.18 | 8,698,892 | 27.75 |
| 1ab | A1 | | | | | | | | | | | |
| HPA- | DN | + | 15,922,432 | 27.12 | 12,571,504 | 27.26 | 13,320,371 | 26.75 | 13,912,265 | 26.57 | 12,028,268 | 27.39 |
| 1ab | A2 | | | | | | | | | | | |
| BG65 | DN | − | −402,292 | 0.00 | −263,831 | 0.00 | −323,044 | 0.00 | −434,705 | 0.00 | −416,573 | 0.00 |
| | A1 | | | | | | | | | | | |
| BG65 | DN | − | −383,518 | 0.00 | −377,673 | 0.00 | −321,353 | 0.00 | −439,087 | 0.00 | −414,986 | 0.00 |
| | A2 | | | | | | | | | | | |

1. A single dry reagent composition prepared by evaporation or air drying for nucleic acid amplification comprising:
at least one nucleic acid amplification enzyme;
at least one corresponding enzyme binder;
a sugar-based stabiliser;
deoxyNucleotide TriphosPhates (dNTPs) or Nucleoside Triphosphates (NTPs) or modified versions thereof;
labelled or unlabeled oligonucleotides for nucleic acid amplification; and
a zwitterionic agent,
wherein the composition excludes at least Tris-based buffers and a nucleic acid template to be amplified.

2. The composition of claim 1, wherein the at least one nucleic acid amplification enzyme is provided in a range from 0.01 to 2 units; and/or
the at least one enzyme binder is provided in a range from 0.01 μg to 1 μg; and/or
the sugar-based stabiliser is provided in a range from 0 to 10% v/v; and/or
the NTPs or DNTPs are each provided in a concentration range from 0.1 mM to 0.4 mM; and/or
the oligonucleotides are provided in a concentration range from 0.001 mM to 110 mM; and/or
the zwitterionic agent is provided in a concentration range from 10 mM to 100 mM.

3. The composition of claim 1, wherein the zwitterionic agent is a 4-(2-hydroxyethyl)-1-piperazineethanesulfonic acid (HEPES) and/or 3-(N-morpholino)propanesulfonic acid (MOPS).

4. (canceled)

5. The composition of claim 1, wherein the at least one nucleic acid amplification enzyme is selected from one or more of: polymerase, DNA polymerase, Taq polymerase, transcriptase and reverse transcriptase, alone or in combination.

6. The composition of claim 1, wherein the at least one enzyme binder is adapted to interact with the at least one corresponding amplification enzyme to temporarily block enzyme activity.

7. The composition of claim 1, wherein the at least one enzyme binder is specific for the at least one nucleic acid amplification enzyme.

8. The composition of claim 1, wherein the at least one enzyme binder is selected from one or more of the following: an antibody, antibody fragment, and synthetic molecule, including aptamers, alone or in combination.

9. The composition of claim 1, wherein the NTPs or dNTPs are modified.

10. The composition of claim 1, wherein the zwitterionic agent is an organic chemical buffer and the composition excludes tricine-based buffers: 3-[(3-cholamidopropyl)dimethylammonio]-1-propanesulfonate(CHAPS), N-Cyclohexyl-2-aminoethanesulfonic acid (CHES) and N-cyclohexyl-2-hydroxyl-3-aminopropanesulfonic acid (CAPSO).

11. The composition of claim 1, where in the enzyme is DNA Taq polymerase.

12. The composition of claim 1 wherein the composition further comprises one or more salts, optionally in the concentration range of 1 to 70 mM, preferably 1-10 mM.

13. The composition of claim 1, wherein the sugar-based stabiliser is selected from a monosaccharide including: Fructose, Galactose, Glucose, Rhamnose and Xylose, a disaccharides including Lactose, Maltose, Trehalose, Sucrose or a Trisaccharide including Melezitose and Raffinose.

14. The composition of claim 1, wherein the composition remains in a stable form, preferably wherein the composition remains in a stable form in a temperature range 0° C. to about 32° C.

15. The composition of claim 1, wherein the composition of the invention is provided in volumes of 5-20 μg, preferably about 10 μg.

16. A process for producing a dry stable single nucleic acid amplification reagent composition comprising:
mixing together non-lyophilised components comprising at least one nucleic acid amplification enzyme, at least one corresponding enzyme binder, a sugar-based stabiliser, Nucleoside Triphosphates or deoxyNucleotideTriphosPhates or modified versions thereof, oligonucleotides and a zwitterionic agent, excluding a Tris-based buffer; and
drying the composition by air drying or evaporation,
wherein the process excludes lyophilisation of the composition.

17. The process for producing a dry stable single nucleic acid amplification reagent composition according to claim 16, wherein the at least one nucleic acid amplification enzyme is provided in a range from 0.01 to 2 units; and/or the at least one enzyme binder is provided in a range from 0.01 μg to 1 μg; and/or the sugar-based stabiliser is provided in a range from 0 to 10% v/v; and/or the Nucleoside Triphosphates or deoxyNucleotideTriphosPhates are each provided in a concentration range from 0.1 mM to 0.4 mM; and/or the oligonucleotides are provided in a concentration range from 0.001 mM to 101 mM; and/or the zwitterionic agent is provided in a concentration range from 10 mM to 100 mM.

18. The process of claim 16, wherein the process excludes a step of mixing a nucleic acid template component.

19. The process of claim 17, wherein the nucleic acid amplification is DNA amplification.

20. The process of claim 16, wherein the at least one enzyme is selected from one or more of: polymerase, DNA polymerase, Taq polymerase, transcriptase and reverse transcriptase, alone or in combination.

21. The process of claim 16, wherein the at least one enzyme binder is specific for the at least one corresponding enzyme.

22. The process of claim 16, wherein the at least one enzyme binder is selected from one or more of an antibody, antibody fragment, or synthetic molecule, including aptamers, alone or in combination.

23. The process of claim 16, wherein the NTPs or dNTPs are modified.

24. The process of claim 16, wherein the buffering agent is an organic chemical buffer further excluding tricine-based buffers:
3-[(3-cholamidopropyl)dimethylammonio]-1-propanesulfonate (CHAPS),
N-Cyclohexylaminoethanesulfonic acid (CHES); and
N-cyclohexyl-2-hydroxyl-3-aminopropanesulfonic acid (CAPSO).

25. The process of claim 16, wherein the zwitterionic agent is a 4-(2-hydroxyethyl)-1-piperazineethanesulfonic acid (HEPES) and optionally the enzyme is DNA Taq polymerase.

26. The process of claim 16, wherein the composition further comprises a salt, preferably a magnesium salt and more preferably Magnesium Chloride.

27. The process of claim 16, wherein the sugar-based stabiliser is selected from a monosaccharide including: Fructose, Galactose, Glucose, Rhamnose and Xylose, a disaccharides including Lactose, Maltose, Trehalose, Sucrose or a Trisaccharide including Melezitose and Raffinose.

28. The process of claim 16, wherein the composition is stable from about −20° C. to about 32° C. and preferably remains stable when stored for 1 to 12 months at that temperature with a desiccant.

29. The process of claim 16, wherein the composition is stable at temperatures of up to 37° C. for at least 3 weeks.

30. The process of claim 16, wherein the drying is by evaporation in a warm environment and/or under vacuum.

31. The process of claim 16, further comprising a step of dividing the composition in to sample volumes of 5-20 μg, preferably volumes of 5-10 μg.
